(19) United States
(12) Patent Application Publication
(10) Pub. No.: US 2019/0223409 A1
(43) Pub. Date: Jul. 25, 2019

(54) ANIMAL THERAPY DEVICE

(71) Applicant: Daniel H. Godfrey, Portland, OR (US)

(72) Inventor: Daniel H. Godfrey, Portland, OR (US)

(21) Appl. No.: 15/490,818

(22) Filed: Apr. 18, 2017

Related U.S. Application Data
(63) Continuation-in-part of application No. 15/132,232, filed on Apr. 18, 2016, now abandoned.
(60) Provisional application No. 62/148,380, filed on Apr. 16, 2015.

(51) Int. Cl. A01K 13/00; A61F 7/08

(52) U.S. Cl. CPC A01K 13/007; A61F 2007/004; A61F 7/08

(57) ABSTRACT

An animal therapy device that allows for the effective pinpoint covering of an injury on the animal and securely holds that covering in place over that injury without causing undue discomfort to the animal and without being easily removed or displaced by the animal. Disclosed embodiments have a base portion sized to encircle a first uninjured portion of the animal, and a covering portion sized to encircle an injured portion of the animal, the covering portion having a neutral position in which it is substantially planar and an engaged position in which it operably conforms around the injured portion of the animal; and, an elongated member extending from the base portion to the covering portion whereby the base portion operably holds the covering portion in place over the injured portion of the animal. A temperature therapy device may be operably secured to the covering portion, and two or more covering portions may be secured to different injured areas on the animal. A second base mounting portion may be provided to further secure the animal therapy device to a second uninjured portion of the animal.

### CROSS-REFERENCE TO RELATED APPLICATIONS

This application is a continuation-in-part of U.S. utility patent application Ser. No. 15/132,232, filed on Apr. 18, 2016, which claims priority to U.S. provisional patent application Ser. No. 62/148,380 filed on Apr. 16, 2015, the disclosures of which are hereby incorporated by reference.

### BACKGROUND

Animals such as dogs and the like enjoy being mobile and active. On occasion, they become injured or undergo medical procedures and require time to heal. In many cases, healing can be facilitated by covering the injured area with a protective covering and/or applying temperature therapy such as a hot/cold pack to the that area. However, many animals tend to seek to remove this covering before its healing effects can be realized.

To date, therapy garments aimed at covering an injured animal have had limited success. In general, these garments tend to be bulky and limit the freedom of movement of the animal, interfere with the animal's ability to perform bodily functions, do not secure effectively to the animal, fail to cover the injured area effectively, are not reversible to allow their use on multiple body parts, and/or fail to allow a temperature therapy device such as a hot/cold pack to be effectively secured over and/or around the injury without the covering be too loose or too tight on the animal.

For example, U.S. Pat. No. 6,240,883 to Gross discloses a protective garment for pets that has two tubular leg sleeves that extend over an animal's left and right front legs with a strap joining them together over the pet's back. One of the sleeves includes a pocket for receiving a hot/cold pack therein. However, the compression sleeves are not adjustable in length or width around the animal's legs. Accordingly, they tend to be too tight or too loose on the pet and limit the sleeve's ability to effectively cover or apply temperature therapy to any area of the pet other than on the front legs immediately adjacent to the pet's body.

Torso covering animal therapy garments tend to wrap around the body of the animal. Examples of these types of garments include U.S. patent application Ser. No. 13/007,954 (Publication No. US2011/0174237) to Sanders-Luckman et al. and U.S. patent application Ser. No. 12/317,174 (Publication No. US2010/0161015) to Wilson et al. In general, these structures limit the areas on the animal where therapy components may be positioned. For example, the hot/cold pack in Sanders-Luckman et al. is limited to under the belly of the animal and is limited to the upper hip region of the animal in Wilson et al. Moreover, since the securing structures in these devices only encircle the animal's torso, the animal's movements, either unintentionally by just moving about, or intentionally by rolling of their back in an effort to remove the garment, can allow the therapy device to be easily moved away from the injured area it is attempting to cover. Tightly synching these torso covering garments around the animal to minimize movement of the therapy device on the animal can also cause undue discomfort to the animal.

### SUMMARY

Thus, despite the known therapy garments for animals, there remains a need for a therapy device that allows for the effective pinpoint covering of an injury on the animal and securely holds that covering in place over that injury without causing undue discomfort to the animal and without being easily removed or displaced by the animal.

Disclosed embodiments include an animal therapy device having a base portion sized to encircle a first portion of the animal, and a covering portion sized to encircle an injured portion of the animal, the covering portion having a neutral position in which it is substantially planar and an engaged position in which it operably conforms around the injured portion of the animal; and, an elongated member extending from the base portion to the covering portion whereby the base portion operably holds the covering portion in place over the injured portion of the animal.

A temperature therapy device may be operably secured to the covering portion, and two or more covering portions may be secured to different injured areas on the animal with a second base mounting portion being provided to further secure the animal therapy device to the animal.

The advantages and features of novelty characterizing aspects of the invention are pointed out with particularity in the appended claims. To gain an improved understanding of the advantages and features of novelty, however, reference may be made to the following descriptive matter and accompanying figures that describe and illustrate various configurations and concepts related to the invention.

### FIGURE DESCRIPTIONS

The foregoing Summary and the following Detailed Description will be better understood when read in conjunction with the accompanying figures.

FIG. 1 is rear isometric view of a first possible animal therapy device in accordance with an embodiment of the present invention showing a possible orientation over the rear hips of an animal.

FIG. 2 is a top plan view of a portion of the animal therapy device of FIG. 1 shown laid open on a substantially flat surface.

FIG. 3 is a top plan view of a stabilizing strap portion of the animal therapy device of FIG. 1 shown laid open on a substantially flat surface.

FIG. 4 is an enlarged, isometric view of the portion of the animal therapy device of FIG. 2 showing possible orientation and placement of therapy devices such as a hot/cold pack.

FIG. 5 is an isometric view of a first possible hot/cold pack for use in the animal therapy device of FIG. 1.

FIG. 6 is a front, plan view of the first possible hot/cold pack of FIG. 5.

FIG. 7 is a top, plan view of the first possible hot/cold pack of FIG. 5.

FIG. 8 is a bottom, plan view of the first possible hot/cold pack of FIG. 5.

FIG. 9 is a right, side view of the first possible hot/cold pack of FIG. 5.

FIG. 10 is an isometric view of a second possible hot/cold pack for use in the animal therapy device of FIG. 1.

FIG. 11 is a front, plan view of the first possible hot/cold pack of FIG. 10.

FIG. 12 is a top, plan view of the first possible hot/cold pack of FIG. 10.

FIG. 13 is a bottom, plan view of the first possible hot/cold pack of FIG. 10.

FIG. 14 is a right, side view of the first possible hot/cold pack of FIG. 10.

FIG. 15 is a side view of a second possible animal therapy device in accordance with an embodiment of the present invention showing a possible orientation over the shoulders of an animal.

FIG. 16 is a front, isometric view of a third possible animal therapy device in accordance with an embodiment of the present invention showing a possible orientation over the carpal area of an animal's front leg.

FIG. 17 is a side view of the third possible animal therapy device of FIG. 16 with a portion cut away to show internal detail.

FIG. 18 is a front, isometric view of a fourth possible animal therapy device in accordance with an embodiment of the present invention showing a possible orientation over the elbow area of an animal's front leg with a portion cut away to show internal detail.

FIG. 19 is a rear, isometric view of a fifth possible animal therapy device in accordance with an embodiment of the present invention showing a possible orientation over the stifle of the animal's hind leg with a portion cut away to show internal detail.

FIG. 20A is the fifth possible animal therapy device of FIG. 19 shown laid open on a substantially flat surface.

FIG. 20B is an alternative possible fifth animal therapy device for use over the stifle of an animal's hind leg with a portion cut away to shown internal detail.

FIG. 21 is a bottom plan view of a portion of the animal therapy device of FIG. 19 showing a possible insertion and orientation of a hot/cold pack received therein.

FIG. 22A is a top plan view of the portion of the animal therapy device of FIG. 19 showing attachment structures in a possible extended position.

FIG. 22B is a top plan view of the portion of the animal therapy device of FIG. 22A showing attachment structures in a possible secure position.

FIG. 23 is a rear, isometric view of a sixth possible animal therapy device in accordance with an embodiment of the present invention showing a possible orientation over a tarsal portion of the animal's rear leg with a portion cut away to show internal detail.

FIG. 24 is a front, isometric view of the third possible animal therapy device of FIG. 16 showing an alternative possible orientation over the carpal area of both an animal's front left and right legs using two covering portions secured so as to also operate as base portions of the other with a portion of one covering portion cut away to show internal detail.

### DETAILED DESCRIPTION

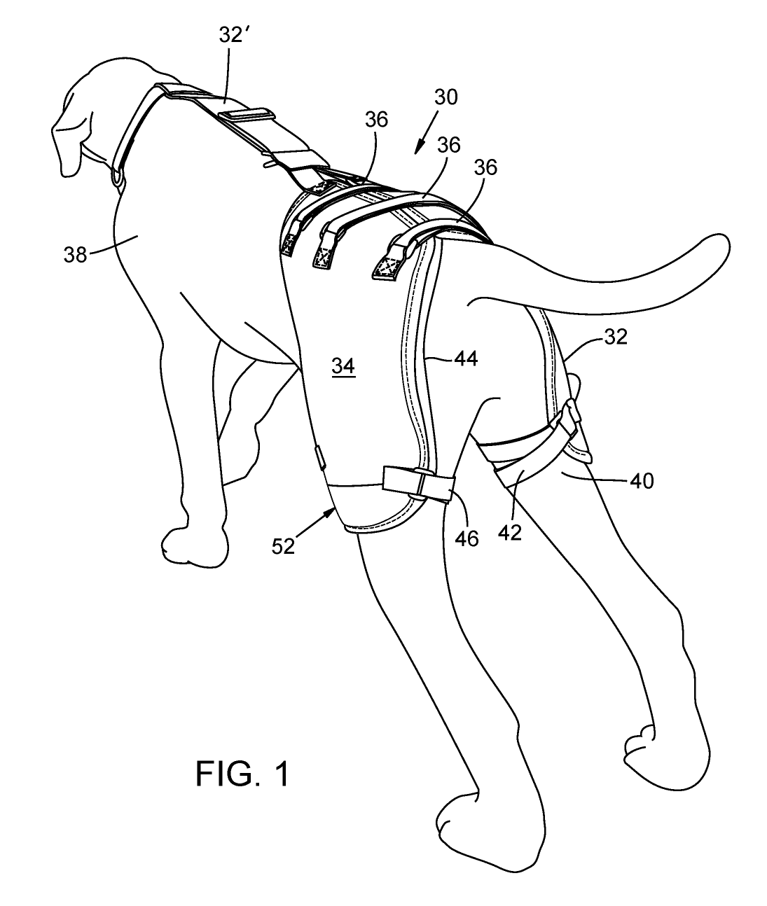

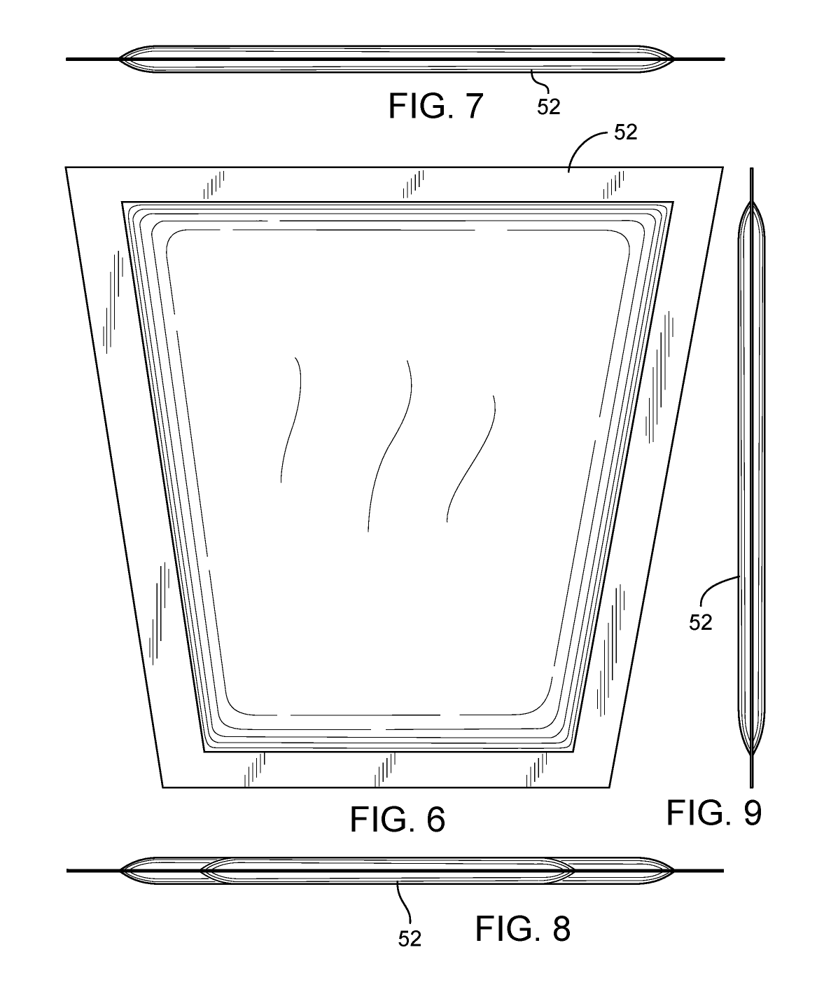

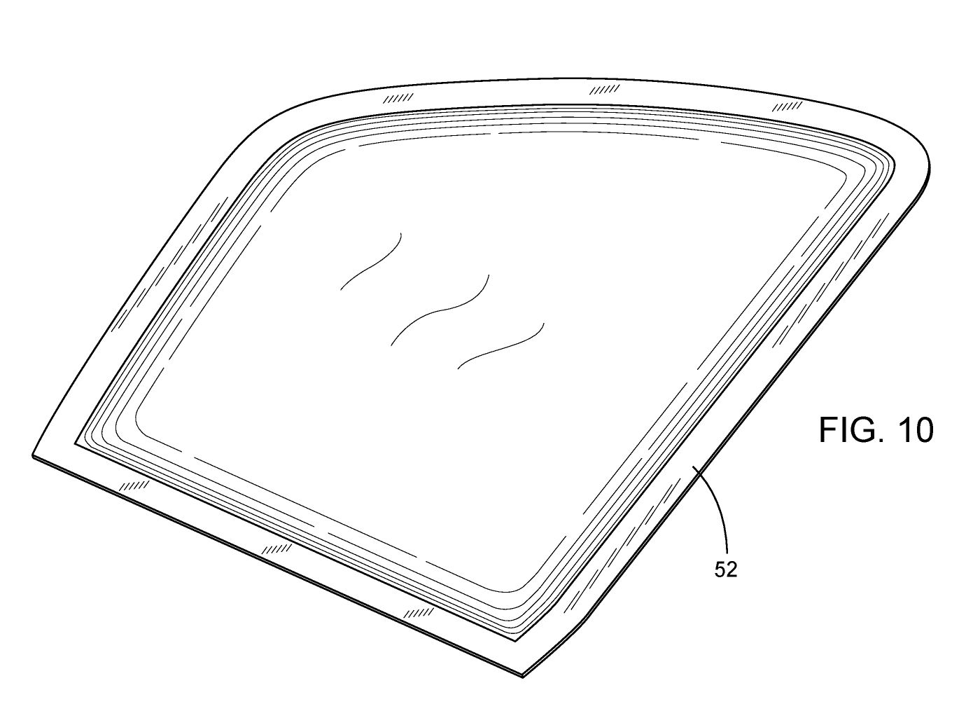

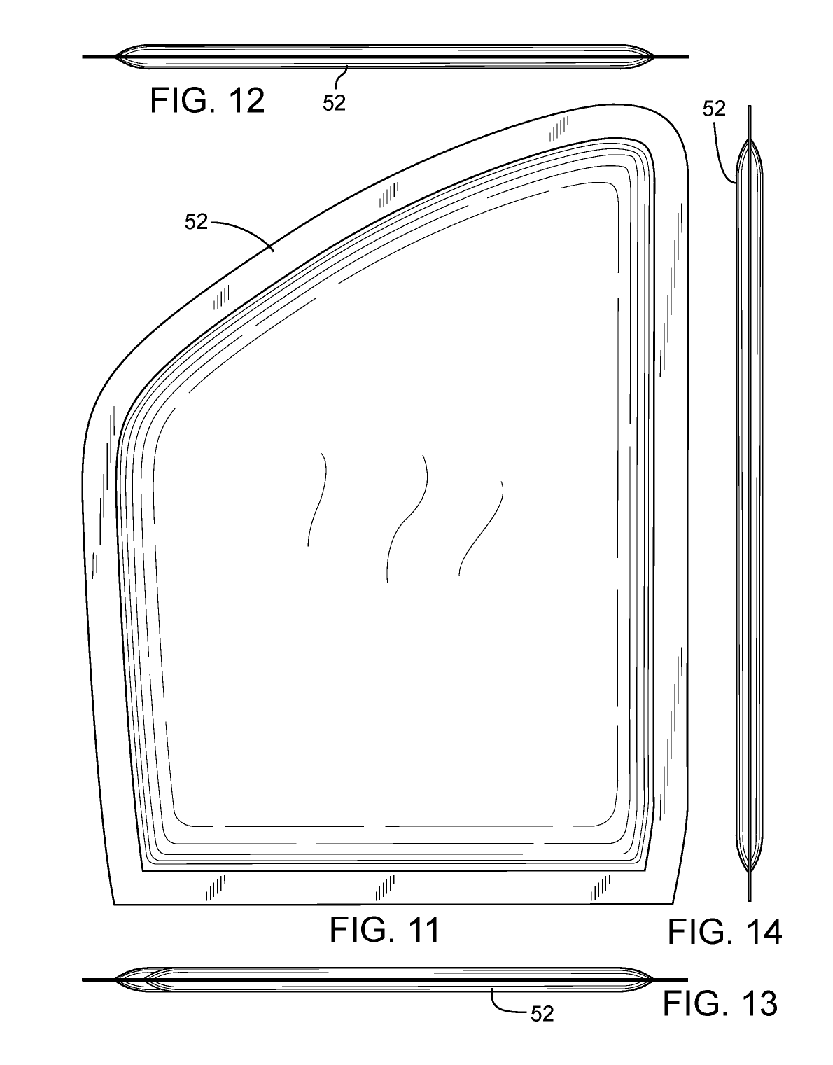

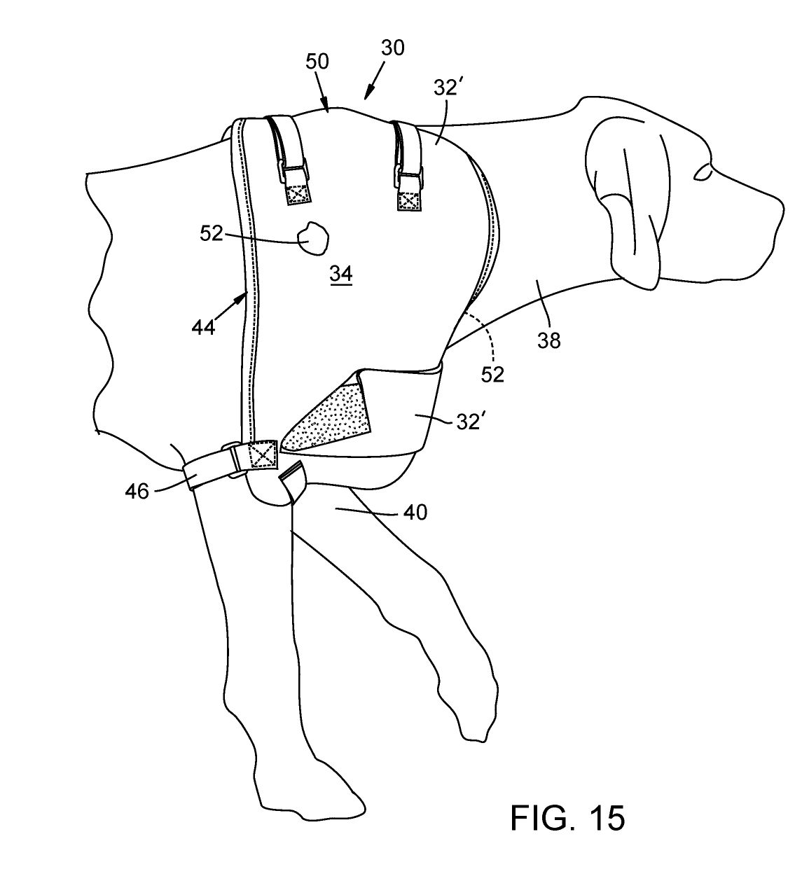

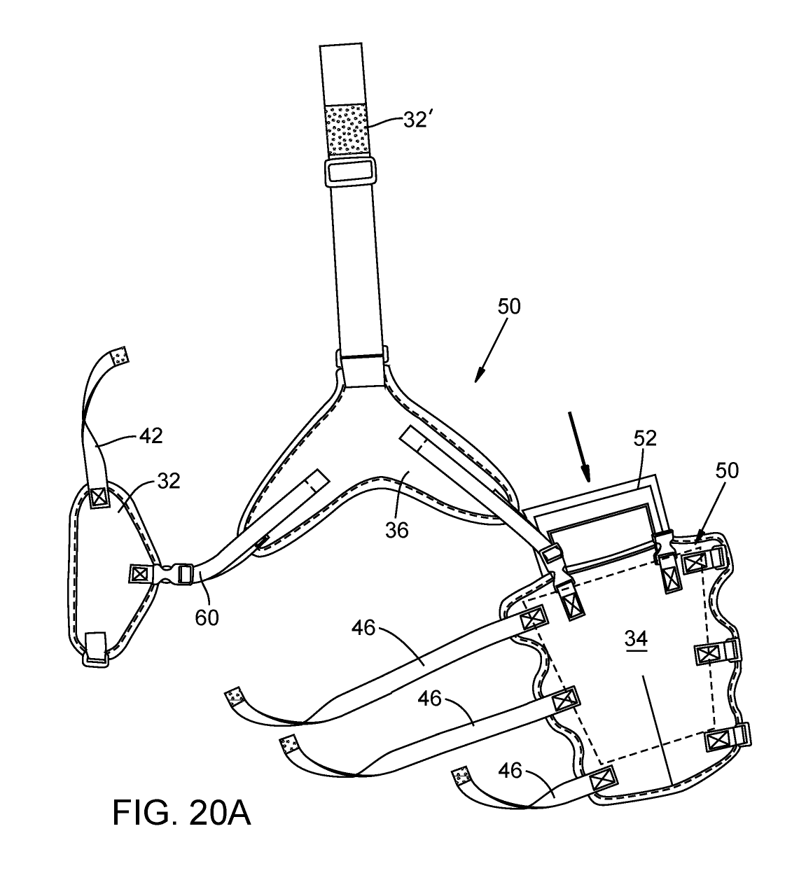

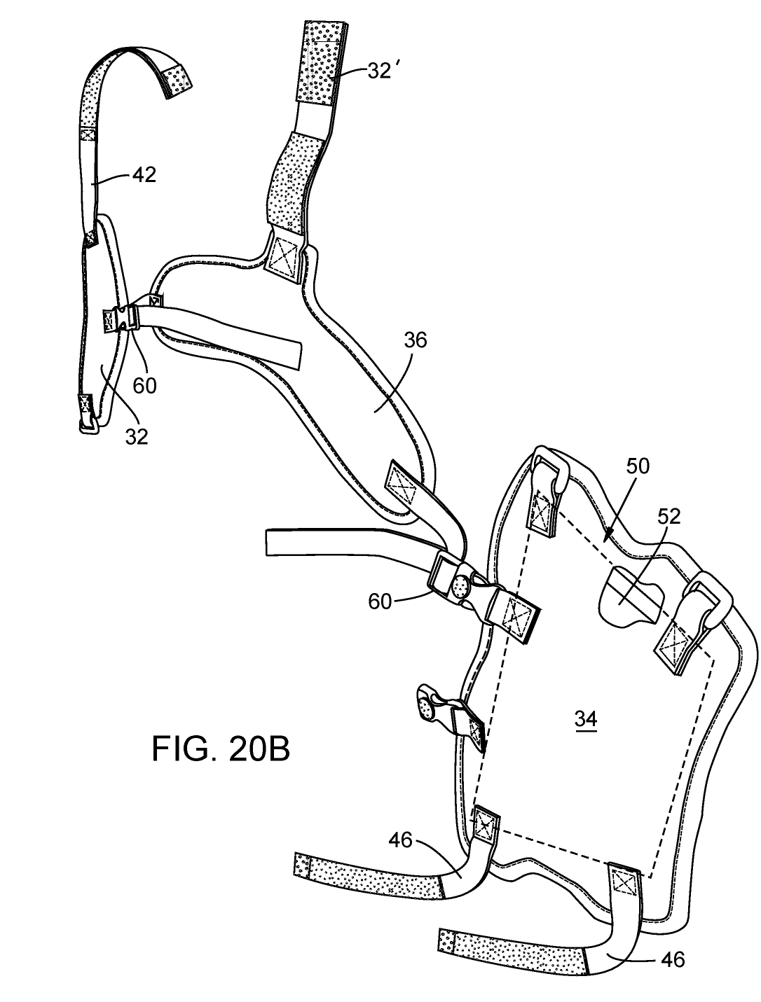

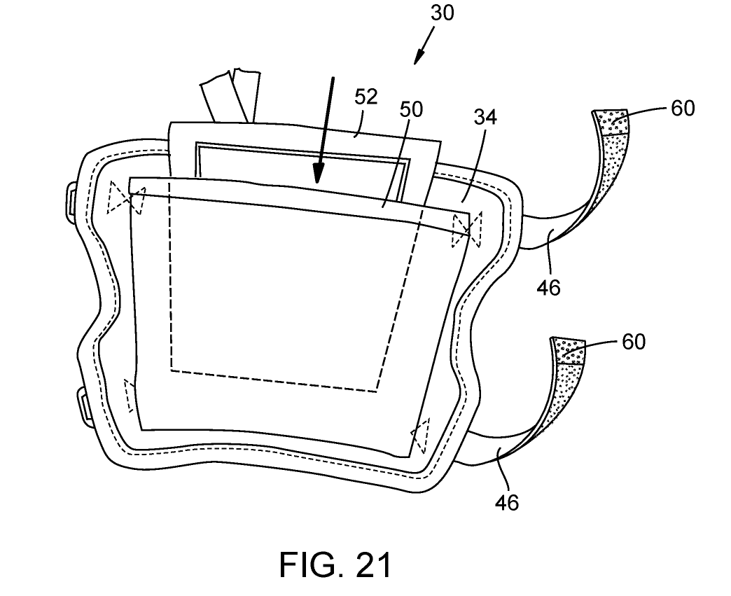

An animal therapy device 30 is shown in FIGS. 1-24. In general, the animal therapy device 30 has the following three components: a base portion 32, a covering portion 34, and an elongated member such as a strap 36 extending between the base portion 32 and covering portion 34. Each of these components is discussed in greater detail below.

The base portion 32 is preferably substantially planar when not in use and is sized to encircle a first portion 40 of the animal, preferably by wrapping around the first portion of the animal. It has a base securing structure 42 detachably securing the base portion to the first portion 40 of the animal. The base securing structure is preferably adjustable, preferably with Velcro or the like, to thereby allow the base portion to be snuggly secured to the first portion of the animal 38 without being too tight or too loose.

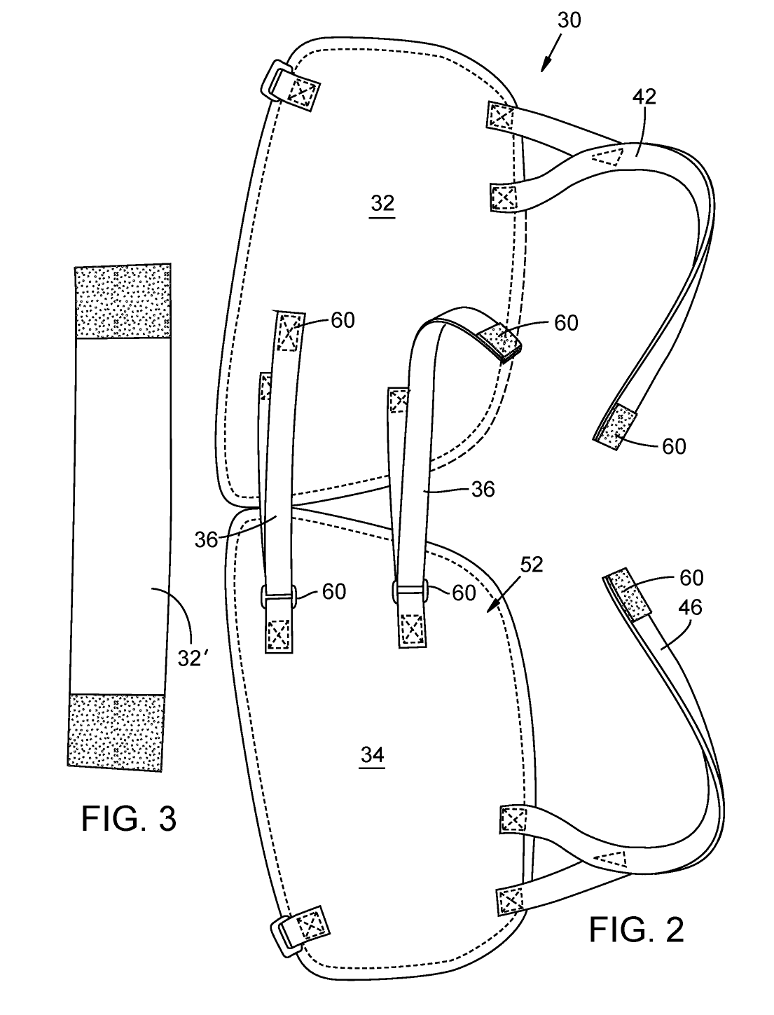

The covering portion 34 is preferably substantially planar when not in use as best shown in FIGS. 2 and 20A-22B, and is sized to cover an injured portion 44 of the animal 38 which is spaced apart from the first portion 40 of the animal 38 to which the base portion 32 is secured. The covering portion 34 is sized and shaped to wrap around a portion of the injured portion 44 of the animal. A cover securing structure 46 detachably secures the covering portion over the injured portion of the animal. The covering portion 43 preferably includes a pocket 50 for receiving a therapy structure such as a hot/cold pack 52 therein. The hot/cold pack 52 is preferably substantially planar and bendable along with the covering portion 34 to wrap around the injured portion of the animal 38 during use.

At least one strap 36 extends from the base portion 32 to the covering portion 34 such that the animal therapy device 30 is secured to the animal 38. Preferably, the strap 36 includes a strap fastening and adjustment structure 60 thereby allowing the position of the base portion 32 and covering portion 34 relative to each other to be adjusted as needed.

More preferably, a second base portion 32′ is detachably secured to a second portion of the animal and operably secured to the covering portion with either the first strap or a second strap such that the animal therapy device is secured to at least three different portions of the animal. For example, the first base portion may be secured around a first leg of an animal, the covering portion may be secured around a second leg of the animal, and the third base portion can be secured around the neck of the animal. Detachably securing the animal therapy devices to three separate portions of the animal greatly improves the hold of the therapy devices over the animal and reduces the possibility of the animal's actions removing or misaligning the device.

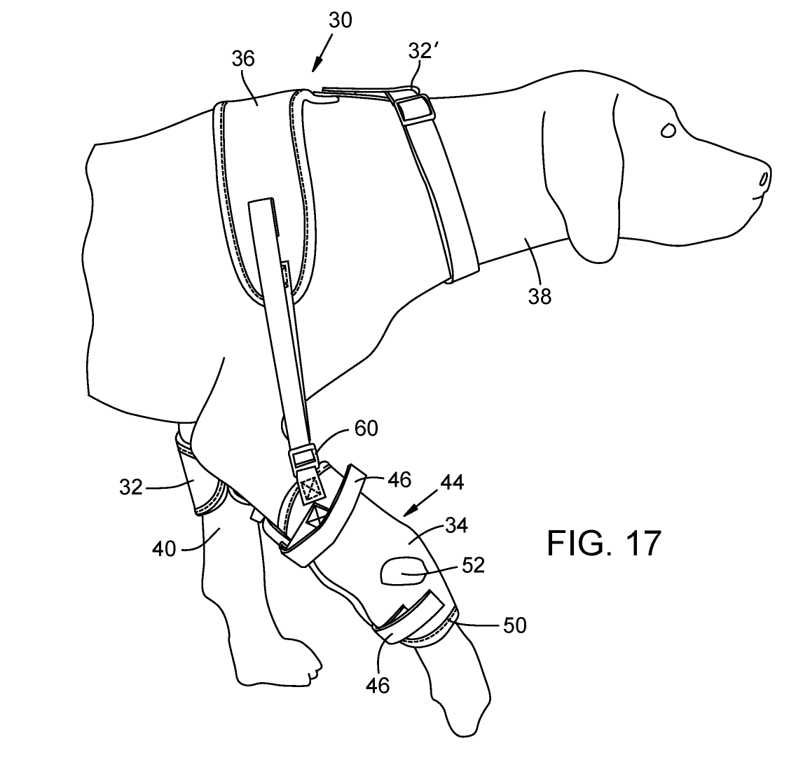

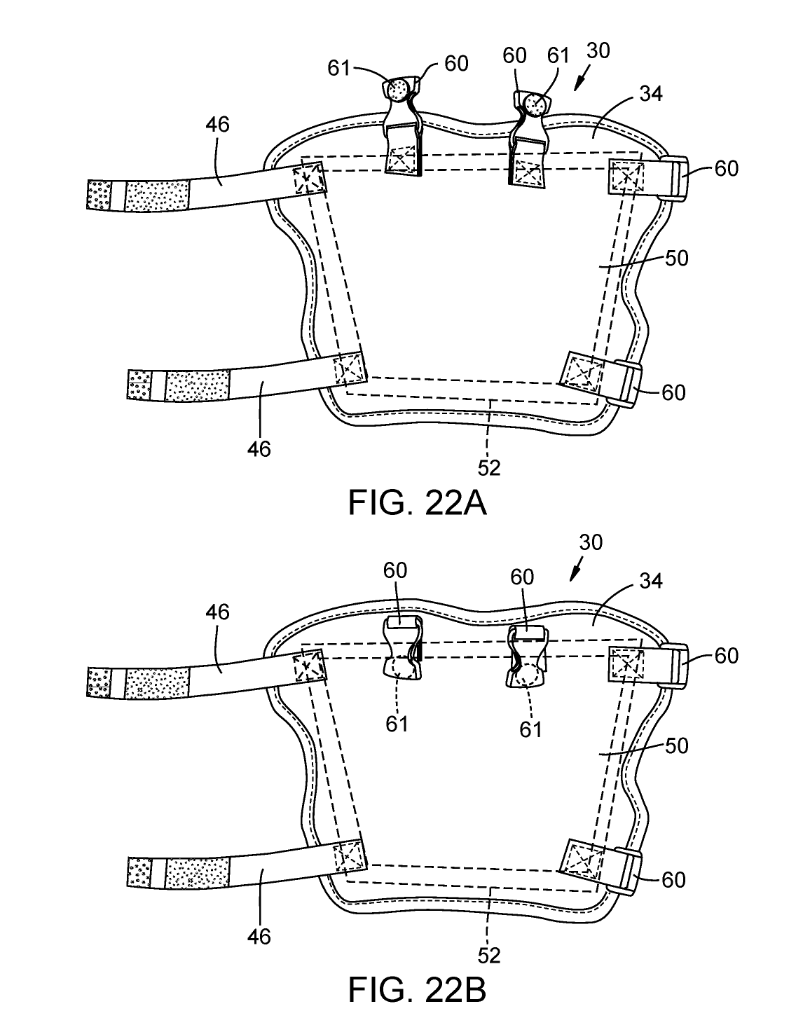

More preferably and as best shown in FIGS. 17 & 22A the base portions and covering portions include a plurality of fastening and adjustment structures 60 positioned so that they can be reversibly positioned and worn on either the left or right portion of the animal. As best shown in FIG. 22B, non-used fastening and adjustment structures 60 for a particular use can be pivoted out of the way and held in place to the exterior of the respective base or covering portion with a fastener 61, such as Velcro or the like, so as to prevent the non-used fastening and adjustment structure 60 from interfering with the animal while worn.

In addition, the base portion and covering portion can be provided in different sizes such as “small,” “medium,” and “large,” to accommodate different sized animals. In such case, it may be desirable to color code the components such as the base and covering portions and/or the related temperature therapy inserts to facilitate their selection and use. For example, “small” components can be colored blue, “medium” components can be colored “red,” and large components can be colored “green,” or the like.

Specific embodiments of the present invention are discussed in greater detail below:

Hip Therapy Wrap

Referring to FIGS. 1-14, an animal therapy device in accordance with the present invention is shown adapted for use over the hips of an animal.

The base portion 32 may be sized to cover one side of the animal's hip and may include a second pocket 50 for operably receiving a therapy item such as a hot/cold pack 52 therein. The base securing structure can include a strap that extends around the animal's first hind leg as shown. An adjustment structure such as a buckle or the like may be provided to allow the strap to snugly but not too tightly secure the base portion to the animal as shown.

The covering portion may be sized to cover the other side of the animal's hip and includes a pocket for operably receding a therapy item such as a hot/cold pack therein. The cover securing structure can include a strap that extends around the animal's second hind leg as shown. An adjustment structure such as a buckle or the like may be provided to allow the strap to snugly but not too tightly secure the covering portion to the animal as shown

Preferably two (FIG. 2) or three (FIG. 1) straps extend between the base portion and covering portion, and each strap includes an adjustment structure 60 such as a cinchable buckle or the like to allow the position of the base portion and covering portion relative to each other to be adjusted as needed.

Preferably a second strap 60 extends from the covering portion and base portion toward the collar of the animal as shown. The second strap includes an adjustment structure such as a buckle or the like to allow the base and covering portions to be snuggly but not too tightly held in place on the animal as shown.

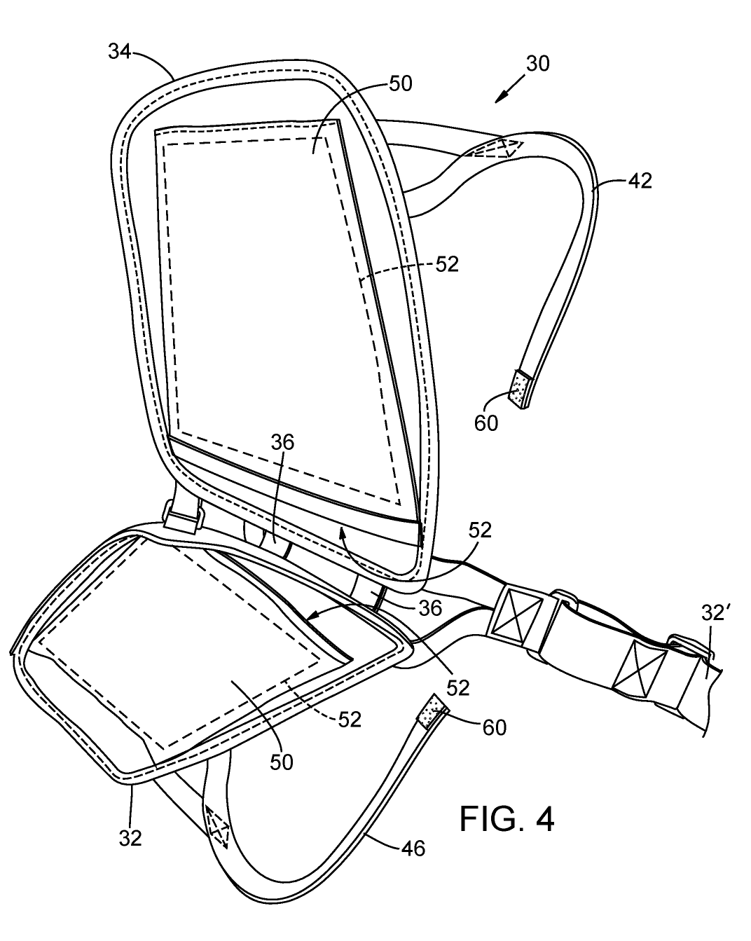

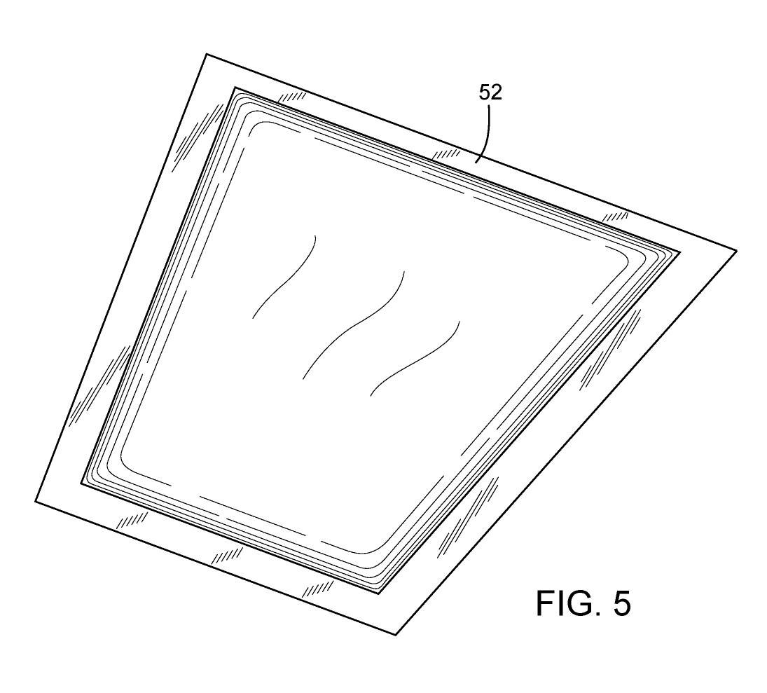

The hot/cold packs 52 of FIGS. 5-14 are sized and shaped to be received within the pockets 50 on the animal therapy device as best shown in FIG. 4. They can be easily inserted into the respective pockets 50 when the cover portion and base portion are in their substantially planar positions shown in FIG. 4. The base and cover portions can then be draped over the animal's hips as best shown in FIG. 1 and secured in place with the straps as previously described.

Shoulder Therapy Wrap

Referring to FIG. 15, an animal therapy device in accordance with the present invention is shown adapted for use over the shoulder or shoulders of an animal. Preferably, two covering portions 34 are provided so as to each cover one of the left or right shoulders of the animal as shown. Each covering portion 34 may include a pocket 50 for receiving a temperature therapy device 52 or the like inside, and also serves as the respective base portion 32 of the other.

Cover securing structures 46 including at least one strap 60 expends between the covering portions 34. A second base portion 32′ may also be provided to further secure the covering portions 34 on the animal.

Carpal Therapy Wrap

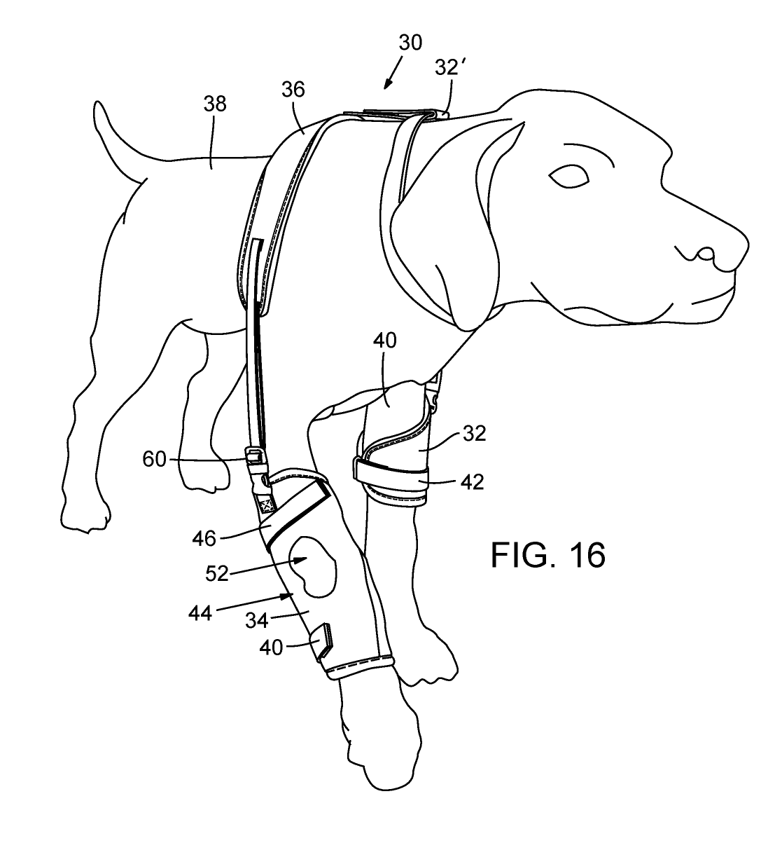

Referring to FIGS. 16 & 17, an animal therapy device in accordance with the present invention is shown adapted for use over the carpal portion of an animal. The covering portion 34 is sized and shaped to encircle the carpal region of an animal's leg as shown. Similarly, the base portion 32 sized and shaped to encircle the opposite leg of the animal. It can be appreciated that the base and covering portions may be worn on opposite legs as needed.

A pocket is provided in the covering portion for receiving a temperature therapy device 52 or the like therein.

Elbow Therapy Wrap

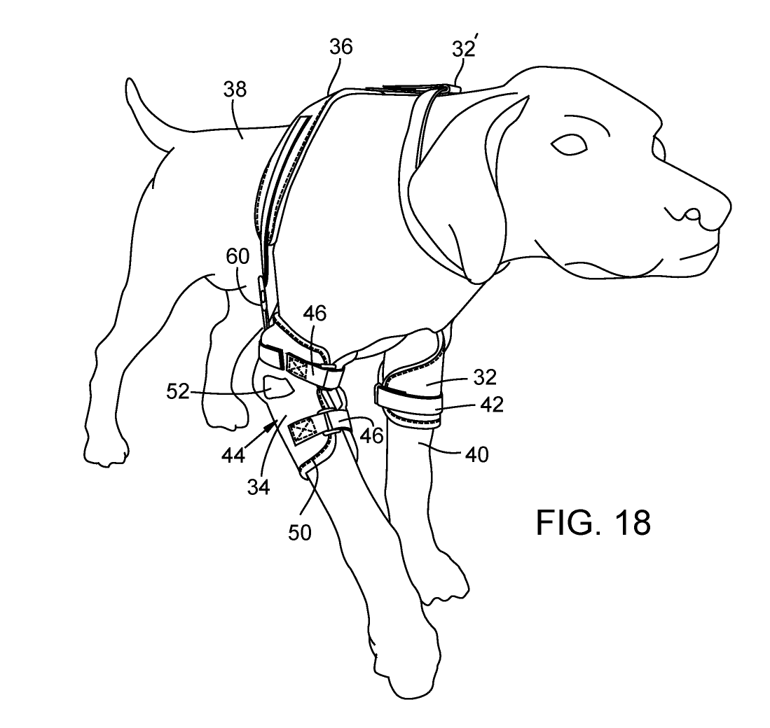

Referring to FIG. 18 an animal therapy device in accordance with the present invention is shown adapted for use over the elbow of an animal.

The covering portion 34 is sized and shaped to encircle the elbow region of an animal's leg as shown. Similarly, the base portion 32 sized and shaped to encircle the opposite leg of the animal. It can be appreciated that the base and covering portions may be worn on opposite legs as needed.

A pocket is provided in the covering portion for receiving a temperature therapy device 52 or the like therein.

Stifle Therapy Wrap

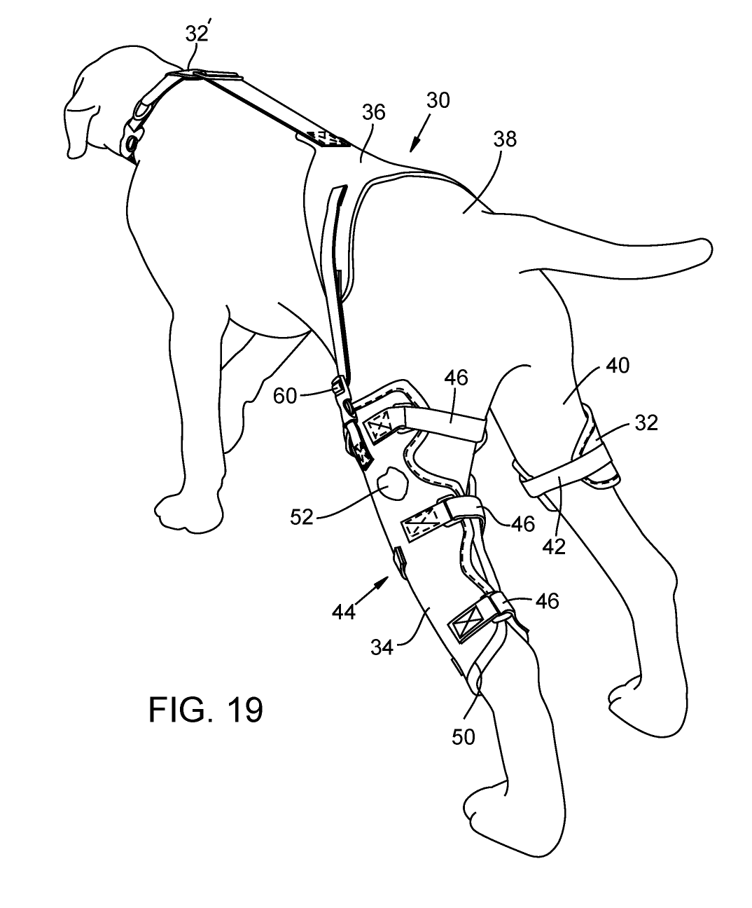

Referring to FIGS. 19-22B, an animal therapy device in accordance with the present invention is shown adapted for use over the stifle portion of an animal.

The covering portion 34 is sized and shaped to encircle the stifle region of an animal's leg as shown. Similarly, the base portion 32 sized and shaped to encircle the opposite leg of the animal. It can be appreciated that the base and covering portions may be worn on opposite legs as needed.

A pocket is provided in the covering portion for receiving a temperature therapy device 52 or the like therein.

Tarsal Therapy Wrap

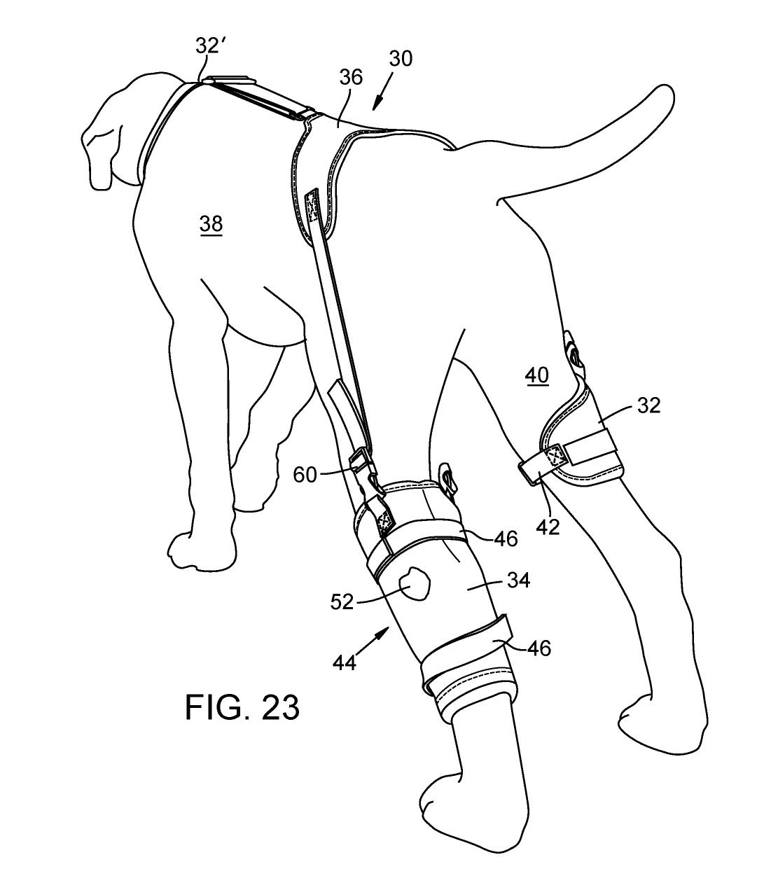

Referring to FIG. 23, an animal therapy device in accordance with the present invention is shown adapted for use over the tarsal of an animal.

The covering portion 34 is sized and shaped to encircle the tarsal region of an animal's leg as shown. Similarly, the base portion 32 sized and shaped to encircle the opposite leg of the animal. It can be appreciated that the base and covering portions may be worn on opposite legs as needed.

A pocket is provided in the covering portion for receiving a temperature therapy device 52 or the like therein.

Dual Covering Portions

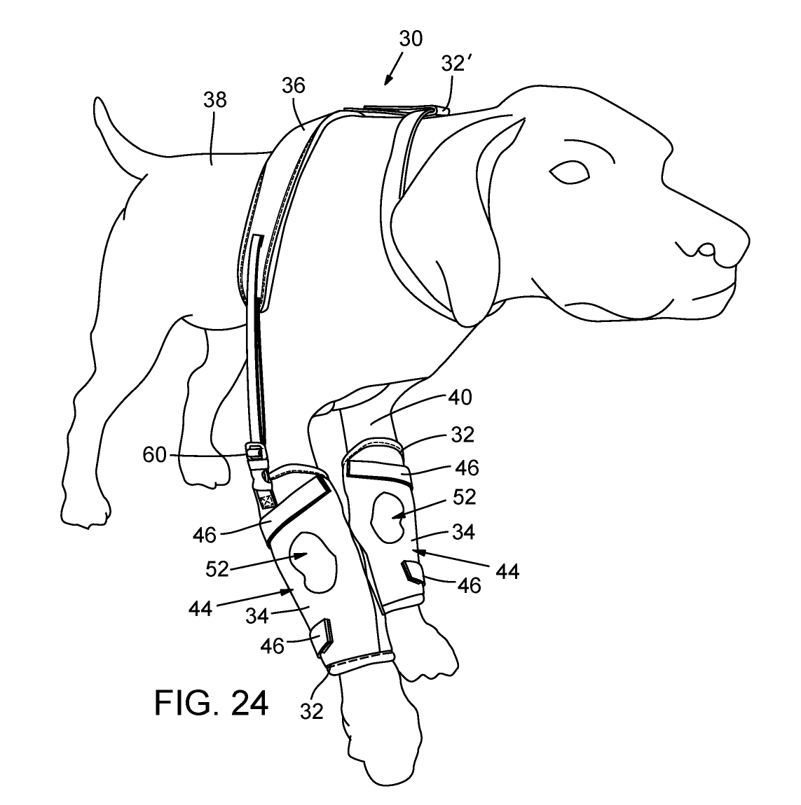

As best shown in FIG. 24, two covering portions 34 may be secured to the animal to provide therapy to two separate regions of the animal. In such case, each covering portion 34 also serves at the base portion 32 for the other as shown.

One skilled in the relevant art will recognize that numerous variations and modifications may be made to the configurations described above. For example, the exemplar in FIG. 24 shows the covering portions 34 covering the carpal portion of the animal's legs, but it can be appreciated that different sized and shaped covering portions as previously described can be secured as needed to cover at least two different areas of the animal. Such modifications and additional embodiments can be made without departing from the scope of the present invention, as defined by the appended claims.

We claim:

1. An animal therapy device comprising:
a base portion sized to encircle a first portion of the animal;
a covering portion sized to encircle an injured portion of the animal, the covering portion having a neutral position in which it is substantially planar and an engaged position in which it operably conforms around the injured portion of the animal; and,
an elongated member extending from the base portion to the covering portion whereby the base portion operably holds the covering portion in place over the injured portion of the animal.

2. The animal therapy device of claim 1, further including:
a second base portion sized to encircle a second portion of the animal, said second base portion operably secured to the base portion and the covering portion thereby providing three secure attachment points on the animal for holding the animal therapy device in place.

3. The animal therapy device of claim 2, further including attachment structures for adjustably securing the base portion, second base portion and covering portion to each other.

4. The animal therapy device of claim 1, wherein:
the base portion includes a first fastener for operably securing the base portion around the first portion of the animal; and,
the covering portion includes a second fastener for operably securing the covering portion around the portion of the first animal limb.

5. The animal therapy device of claim 4, wherein the elongate member includes an adjustment structure for adjusting the length of the elongate member.

6. The animal therapy device of claim 1, further including a temperature therapy device operably secured to the covering portion, said temperature therapy device substantially planar when the covering portion is in said neutral position.

7. The animal therapy device of claim 6, wherein the covering portion includes a pocket for operably receiving the temperature therapy device therein such that the temperature therapy device operably engages the injured portion of the animal when the covering portion is in the said engaged position.

8. The animal therapy device of claim 1, wherein the first animal portion is a first limb of the animal and the injured animal portion is a second limb of the animal.

9. The animal therapy device of claim 1, wherein the first animal portion is the neck of the animal.

10. The animal therapy device of claim 2, wherein the first animal portion is a first limb of the animal, the second portion of the animal is the neck portion of the animal, and the injured portion of the animal is a second limb of the animal.

11. The animal therapy device of claim 10, wherein the second limb of the animal includes the covering portion covering a portion of the second limb selected from the group consisting of the carpal, elbow, stifle and tarsal of the second limb.

12. The animal therapy device of claim 1, wherein the first animal portion is selected from the group consisting of the first hip and first shoulder of the animal, and the injured portion of the animal is selected from the group consisting of the second hip and second shoulder of the animal.

13. The animal therapy device of claim 12, wherein the second animal portion is selected from the group consisting of the neck and chest of the animal.

14. The animal therapy device of claim 1, further including a plurality of covering portions each having a different size to accommodate different sized animals.

15. The animal therapy device of claim 14, wherein the covering portions are color coded by size.

16. The animal therapy device of claim 6, further including a plurality of covering portions each having a different size to accommodate different sized animals and a plurality of temperature therapy devices each having a different size to be operably received in the different sized covering portions.

17. The animal therapy device of claim 16, wherein the temperature therapy devices are color coded by size.

18. The animal therapy device of claim 1, wherein the covering portion includes a plurality of attachment structures for operably engaging the elongate member, thereby allowing the covering portion to be operably secured to either the left of right side on the animal and defining used attachment structures and unused attachment structures.

19. The animal therapy device of claim 18, further including a fastener for detachably securing unused attachment structures to the covering portion thereby preventing said unused attachment structures from interfering with the animal when the covering portion is in the engaged position on the animal.
